(19) United States
(12) Patent Application Publication
(10) Pub. No.: US 2009/0205663 A1
(43) Pub. Date: Aug. 20, 2009

(54) CONFIGURING THE OPERATION OF AN ALTERNATING PRESSURE VENTILATION MODE

(75) Inventors: Joseph Douglas Vandine, Newark, CA (US); Gary Scott Milne, Louisville, CO (US)

Correspondence Address: NELLCOR PURITAN BENNETT LLC;ATTN: IP LEGAL, 60 MIDDLETOWN AVENUE, NORTH HAVEN, CT, 06473, US

(73) Assignee: Nellcor Puritan Bennett LLC, Boulder, CO (US)

(21) Appl. No.: 12/367,332

(22) Filed: Feb. 6, 2009

Related U.S. Application Data
(60) Provisional application No. 61/029,894, filed on Feb. 19, 2008.

(51) Int. Cl. A61M 16/00

(52) U.S. Cl. 128/204.23; 128/204.26

(57) ABSTRACT

Systems and methods for configuring the operation of an alternating pressure ventilation mode are provided. According to one embodiment a configuration method includes monitoring gas flow between a patient and a ventilation system. Based on the monitoring, a peak expiratory flow rate (PEFR) is determined Information indicative of values of parameters of the ventilation mode are received, including a higher pressure setting, a lower pressure setting and a duration of the higher pressure setting. User input is also received indicative of a target percentage of PEFR at which the ventilation system should cycle from the lower pressure setting to the higher pressure setting. Based on the target percentage, a duration of the lower pressure setting is programmatically determined. Finally, the ventilation system is configured to automatically cycle between the higher and lower pressure setting at a predetermined flow based on the parameters and the duration of the lower pressure setting.

### RELATED APPLICATION

This application claims priority from U.S. patent application Ser. No. 61/029,894 which was filed on Feb. 19, 2008, and is incorporated herein by reference in its entirety.

### BACKGROUND OF THE INVENTION

Embodiments of the present invention generally relate to mechanical ventilation, and more particularly to systems and methods for configuring the operation of an alternating pressure ventilation mode in support of various ventilation strategies, such as BiLevel ventilation or Airway Pressure Release Ventilation (APRV).

Modern ventilators are designed to ventilate a patient's lungs with gas, and to thereby assist the patient when the patient's ability to breathe on their own is somehow impaired. Increased clinical focus on recruitment of functional lung in various disease states has created a high degree of interest in using alternating pressure ventilation. As used herein, the phrase “alternating pressure ventilation” generally refers to a form of augmented pressure ventilation in which the lungs are maintained in a distended state by a mechanical ventilator sufficient to keep recruitable alveoli open, but ventilation is augmented by periodically releasing pressure to a lower level to allow better clearance of alveolar carbon dioxide. During alternating pressure ventilation, two different levels of Positive End-Expiratory Pressure (PEEP) are applied to the airways and alveoli in alternating fashion to maintain a certain residual amount of air in the lungs, thereby preventing complete emptying on exhalation and avoiding airway collapse.

Various ventilatory strategies are available within alternating pressure ventilation, such as BiLevel ventilation and APRV. BiLevel ventilation and APRV are differentiated by the time allowed at the lower PEEP level (PEEP_{LOW}). If the time spent at both the upper PEEP level (PEEP_{HI}) and the lower PEEP level is long enough to allow spontaneous breathing at both levels, the ventilatory strategy is commonly referred to as BiLevel; whereas APRV implies a short duration at the lower PEEP level, in which all spontaneous breathing takes place at the upper PEEP level.

Turning now to FIG. 1, an airway pressure versus time tracing 100 and a corresponding inspiratory and expiratory gas flow versus time tracing 105 for an alternating pressure ventilation mode are depicted. Referring to the airway pressure versus time tracing 100, two phases are readily identifiable, a higher positive pressure phase 110 and a release phase 120. According to the present example, during the higher positive pressure phase 110 a continuous positive airway pressure (CPAP) level of approximately 17 cmH₂O (PEEP_{HI} 140) is applied for a duration referred to as T_{HIGH} 145. The positive pressure phase 110 is followed by the release phase 120, in which the pressure is released to some lower level, typically between 0-5 cmH₂O (PEEP_{LOW} 130). The duration of the release phase 120 is referred to as T_{LOW} 135.

The periodicity of transition of alternating pressure ventilation is defined by selecting the duration (T_{HIGH} 145) that airway pressure should be at PEEP_{HI} 140 and the duration (T_{LOW} 135) that the pressure should be allowed to remain at PEEP_{LOW} 130. Consequently, existing ventilation systems require at least four inputs (i.e., the value of PEEP_{HI} 140, the value of PEEP_{LOW} 130, the value of T_{HIGH} 145 and the value of T_{LOW} 135) from the clinician to appropriately configure an alternating pressure ventilation mode, such as APRV. Notably, however, in the context of APRV, there is currently no consensus regarding an appropriate value of T_{LOW} 135.

While there is no consensus regarding the absolute duration of time that the pressure should remain at PEEP_{LOW} 130, there is a growing school of thought that suggests the end of the release phase 120 (and hence the beginning of the next positive pressure phase 110) should be at a point defined in terms of a target percentage of the peak observed expiratory flow rate. With reference to both time tracings 100 and 105, the peak expiratory flow rate (PEFR) 150 is observed at the transition point from PEEP_{HI} 140 to PEEP_{LOW} 130; and the point at which the flow of gas from the patient's lungs reaches the desired target percentage of the PEFR 150 is referred to as the target percentage of PEFR 160.

Thus, to appropriately configure an APRV mode of current ventilation systems, clinicians must estimate both the point in the lung flow function that most closely approximates their target (i.e., target percentage of PEFR 160) as well as the amount of time it took to achieve this estimated target from the beginning of the release phase 120. Then, based on these estimates, the clinician is required to manually input the value of T_{LOW} 135 that is to he used by the ventilation system to trigger future transitions from the lower pressure setting to the higher pressure setting.

At least one drawback of this current approach of configuring an APRV mode is that the timing at which the target percentage of PEFR 160 occurs varies over time based on the condition of the patient's lungs. As a result, over time, a fixed time value for T_{LOW} 135 manually estimated by the clinician may no longer achieve the desired physiologic response due to changing lung dynamics. As a result, the clinician must re-estimate and re-enter the value on a periodic basis.

### BRIEF SUMMARY OF THE INVENTION

Systems and methods are described for configuring the operation of an alternating pressure ventilation mode. According to one embodiment, a method is provided for controlling a ventilation system. A flow of gas between a patient and the ventilation system is monitored. Based on the monitoring, a peak expiratory flow rate (PEFR) is determined. Information indicative of values of a number of parameters of an alternating pressure ventilation mode of the ventilation system are received, including at least a higher pressure setting, a lower pressure setting and a duration of the higher pressure setting. User input is also received indicative of a desired percentage of the PEER at which the ventilator system should cycle from the lower pressure setting to the higher pressure setting. Based on the desired percentage of the PEFR, a duration of the lower pressure setting is programmatically determined. Finally, the ventilation system is configured to automatically cycle between the higher pressure setting and the lower pressure setting at a pre-determined flow based on the plurality of parameters and the duration of the lower pressure setting.

In the aforementioned embodiment, the alternating pressure ventilation mode may represent an Airway Pressure Release Ventilation (APRV) mode in which a ratio of the duration of the higher pressure setting to the duration of the lower pressure setting is such that all spontaneous breathing by the patient takes place during the higher pressure setting. Alternatively, in the aforementioned embodiment, the alternating pressure ventilation mode may represent a BiLevel ventilation mode in which a ratio of the duration of the higher pressure setting to the duration of the lower pressure setting is configured to allow spontaneous breathing by the patient during both the lower pressure setting and the higher pressure setting.

In various instances of the aforementioned embodiments, the gas flow monitoring includes metering a flow of breathing gas delivered to the patient from the ventilation system via a first flow sensor as well as metering expiratory gas flow returning from the patient to the ventilation system via a second flow sensor.

In the context of various of the aforementioned embodiments, the gas flow monitoring may include metering both a flow of breathing gas delivered to the patient by the ventilation system and a flow of gas returning from the patient to the ventilation system by a single sensor positioned at a port defining an entry to an airway of the patient.

In various instances of the aforementioned embodiments, receiving information regarding the parameter values involves receiving predefined default parameter values from a ventilation mode profile. Alternatively, a subset of parameter values are provided as user input via a user interface of the ventilation system; and the remainder of the parameter values are predefined default parameter values associated with a ventilation mode profile.

In the aforementioned embodiment, the user input indicative of a desired percentage of the PEFR may include touch screen input associated with an inspiratory and expiratory gas flow versus time tracing depicted on a user interface of the ventilation system. Alternatively, the user input indicative of a desired percentage of the PEFR includes a user selection from a predefined set or range of PEFR percentages displayed to the user via a user interface of the ventilation system. Furthermore, the predefined set or range of PEFR percentages may be limited to values between approximately 20% of PEFR and approximately 75% of PEFR. The user input indicative of a desired percentage of the PEFR may also be provided in the form of numerical input. In such circumstances, a user interface of the ventilation system may alert the user when the numerical input is outside a range of approximately 20 to approximately 75.

Other embodiments of the present invention provide a ventilation system, which includes a gas flow path, a pressure controller, one or more flow sensors, a user interface, a processor and a computer-readable medium. The gas flow path is to deliver breathing gas from a gas source to a patient. The pressure controller is located along the gas flow path and configured to cycle the ventilation system among a plurality of pressure settings. The one or more flow sensors are located along the gas flow path and are configured to monitor a flow of gas between the patient and the ventilation system. The user interface is configured to display information to an end user of the ventilation system regarding airway pressure of the patient and the flow of gas and to receive information from the end user indicative of one or more values of parameters associated with an alternating pressure ventilation mode of the ventilation system or from which the one or more values can be derived. The computer-readable medium has stored thereon instructions executable by the processor, which cause the processor to receive information from the one or more flow sensors regarding the flow of gas; determine a peak expiratory flow rate (PEFR) based on the information regarding the flow of gas; receive values for a subset of the parameters associated with the alternating pressure ventilation mode, including a higher pressure setting, a lower pressure setting and a duration of the higher pressure setting; receive user input via the user interface indicative of a desired percentage of the PEFR at which the ventilator system should cycle from the lower pressure setting to the higher pressure setting; programmatically determine a duration of the lower pressure setting based on the desired percentage of the PEFR; and cause the ventilation system to automatically cycle between the higher pressure setting and the lower pressure setting at a predetermined flow by conveying the higher pressure setting, the lower pressure setting, the duration of the higher pressure setting and the duration of the lower pressure setting to the pressure controller.

In some instances of the aforementioned embodiment, the ventilation system is a critical care ventilator

In various instances of the aforementioned embodiment, the alternating pressure ventilation mode is an Airway Pressure Release Ventilation (APRV) mode or a BiLevel ventilation mode.

In the aforementioned embodiment, the one or more flow sensors may include two sensors, a first sensor configured to meter a flow of breathing gas delivered to the patient from the ventilation system and a second sensor configured to meter expiratory gas flow returning from the patient to the ventilation system. Alternatively, a single flow sensor may be positioned at a port defining an entry to an airway of the patient and this single flow sensor may meter both a flow of breathing gas delivered to the patient by the ventilation system and a flow of gas returning from the patient to the ventilation system.

According to one embodiment, yet another method is provided for controlling a ventilation system, including a step for monitoring a flow of gas between a patient and the ventilation system; a step for determining a peak expiratory flow rate (PEFR) based on the monitoring; a step for receiving information indicative of values of multiple parameters of an alternating pressure ventilation mode of the ventilation system, including at least a higher pressure setting, a lower pressure setting and a duration of the higher pressure setting; a step for programmatically determining a duration of the lower pressure setting based on user input indicative of a percentage of the PEFR at which the user desires the ventilation system to transition from the lower pressure setting to the higher pressure setting; and a step for configuring the ventilation system to automatically cycle between the higher pressure setting and the lower pressure setting at a pre-determined time based on the plurality of parameters and the duration of the higher pressure setting.

In various instances of the aforementioned embodiment, the alternating pressure ventilation mode may be selected from multiple alternating pressure ventilation modes supported by the ventilation system, including one or more of an Airway Pressure Release Ventilation (APRV) mode and a BiLevel ventilation mode.

This summary provides only a general outline of some embodiments of the invention. Many other objects, features, advantages and other embodiments of the invention will become more fully apparent from the following detailed description, the appended claims and the accompanying drawings.

### BRIEF DESCRIPTION OF THE DRAWINGS

A further understanding of the various embodiments of the present invention may be realized by reference to the figures which are described in remaining portions of the specification. In the figures, like reference numerals may be used throughout several of the figures to refer to similar components. In some instances, a sub-label consisting of a lower case letter is associated with a reference numeral to denote one of multiple similar components. When reference is made to a reference numeral without specification to an existing sub-label, it is intended to refer to all such multiple similar components.

FIG. 1 depicts an airway pressure versus time tracing and a corresponding inspiratoiy and expiratory gas flow versus time tracing for an alternating pressure ventilation mode;

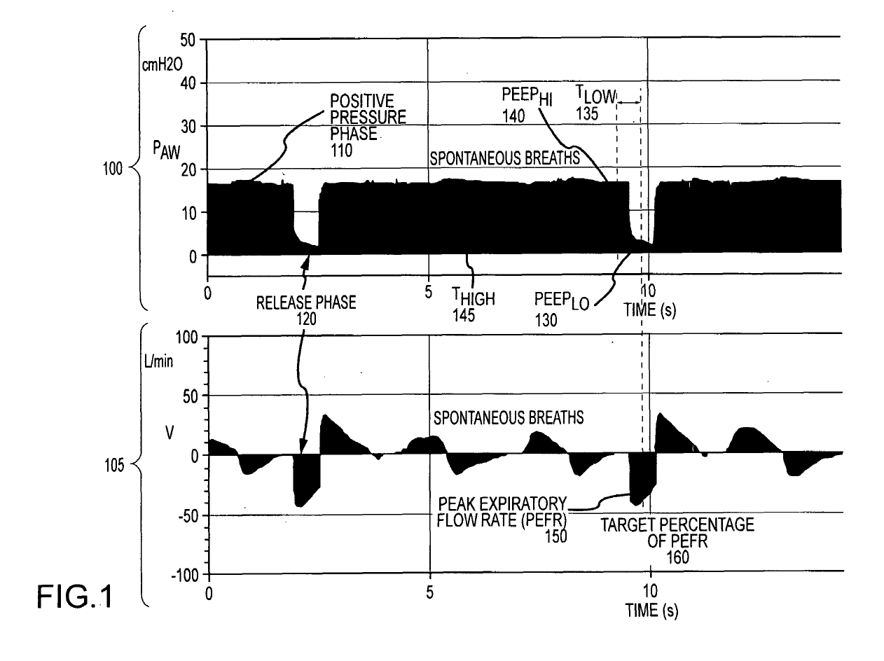

FIG. 2 is a simplified block diagram of a ventilation system in accordance with an embodiment of the present invention;

FIG. 3 depicts a ventilator control system in accordance with an embodiment of the present invention; and

FIG. 4 is a flow diagram illustrating alternating pressure ventilation mode configuration in accordance with an embodiment of the present invention.

### DETAILED DESCRIPTION OF THE INVENTION

Systems and methods are described for configuring the operation of an alternating pressure ventilation mode. Increased clinical focus on recruitment of functional lung in various disease states has created a high degree of interest in using inverse inspiratory to expiratory time ratio (I:E ratio) alternating pressure ventilation modes. Such ventilation strategies are focused on maintaining the lungs in a distended state sufficient to keep all recruitable alveoli open, but to augment ventilation by periodically releasing pressure to allow better clearance of alveolar carbon dioxide. Various embodiments of the present invention provide an improved ventilation system user interface that both simplifies initiation of an alternating pressure ventilation mode and maintains the optimality of T_{LOW}. In one embodiment of the present invention, rather than requiring the clinician to estimate T_{LOW} based on the clinician's desired target percentage of PEFR, the clinician may directly input information indicative of the target percentage of PEFR at which the clinician would like the ventilation system to cycle from PEEP_{LOW} to PEEP_{HI}. The ventilation control system may then automatically calculate the appropriate T_{LOW} value based on the desired target and input from one or more flow sensors of the ventilation system. Furthermore, the ventilation control system may subsequently recalculate T_{LOW} on a periodic basis based on the configured target percentage of PEFR and the ongoing monitoring of gas flow between the patient and the ventilation system. Advantageously, in this manner, the clinician's intent with respect to operation of the alternating pressure ventilation mode and the optimality of T_{LOW} may be maintained despite fluctuations in the patient's lung time constant, which varies as the patient's lung condition improves or deteriorates.

In the following description, for the purposes of explanation, numerous specific details are set forth in order to provide a thorough understanding of embodiments of the present invention. It will be apparent, however, to one skilled in the art that embodiments of the present invention may be practiced without some of these specific details.

Embodiments of the present invention may include various steps, which will be described below. The steps may be performed by hardware components or may be embodied in machine-executable instructions, such as firmware or software, which may be used to cause a general-purpose or special-purpose processor programmed with the instructions to perform the steps. Alternatively, the steps may be performed by a combination of hardware, software, firmware and/or one or more human operators, such as a clinician.

Embodiments of the present invention may be provided as a computer program product which may include a machine-readable medium having stored thereon instructions which may be used to program a processor associated with a ventilation control system to perform various processing. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, MultiMedia Cards (MMCs), secure digital (SD) cards, such as miniSD and microSD cards, or other type of media/machine-readable medium suitable for storing electronic instructions. Moreover, embodiments of the present invention may also be downloaded as a computer program product. The computer program may be transferred from a remote computer to a requesting computer by way of data signals embodied in a carrier wave or other propagation medium via a communication link (e.g., a modem or network connection). For example, various subsets of the functionality described herein may be provided within a legacy or upgradable ventilation system as a result of installation of a software option or performance of a fiirmware upgrade.

While, for convenience, various embodiments of the present invention may be described with reference to a particular alternating pressure ventilation mode, such as APRV mode, the present invention is also applicable to various other alternating pressure ventilation modes, such as BiLevel ventilation modes and the like.

As used herein, the phrase “alternating pressure ventilation mode” is used in its broadest sense to refer to any ventilation mode that cycles between a higher pressure level and a lover pressure level. For purposes of this definition, the time spent at either level or the specific T_{HI}:T_{LOW} (time high to time low ratio) is of no consequence. Thus, an alternating pressure ventilation mode may include, but is not limited to, (i) an Airway Pressure Release Ventilation (APRV) mode in which a ratio of the duration of the higher pressure setting to the duration of the lower pressure setting is such that all spontaneous breathing by the patient takes place during the higher pressure setting; and (ii) a ventilation mode in which a ratio of the duration of the higher pressure setting to the duration of the lower pressure setting is configured to allow spontaneous breathing by the patient during both the lower pressure setting and the higher pressure setting.

As used herein, the terms “connected” or “coupled” and related terms are used in an operational sense and are not necessarily limited to a direct physical connection or coupling. Thus, for example, two devices of functional units may be coupled directly, or via one or more intermediary media or devices. As another example, devices or functional units may be coupled in such a way that information can be passed there between, while not sharing any physical connection one with another. Based on the disclosure provided herein, one of ordinary skill in the art will appreciate a variety of ways in which connection or coupling exists in accordance with the aforementioned definition.

As used herein, the phrases “in one embodiment,” “according to one embodiment,” and the like generally mean the particular feature, structure, or characteristic following the phrase is included in at least one embodiment of the present invention, and may be included in more than one embodiment of the present invention. Importantly, such phases do not necessarily refer to the same embodiment. If the specification states a component or feature “may”, “can”, “could”, or “might” be included or have a characteristic, that particular component or feature is not required to be included or have the characteristic.

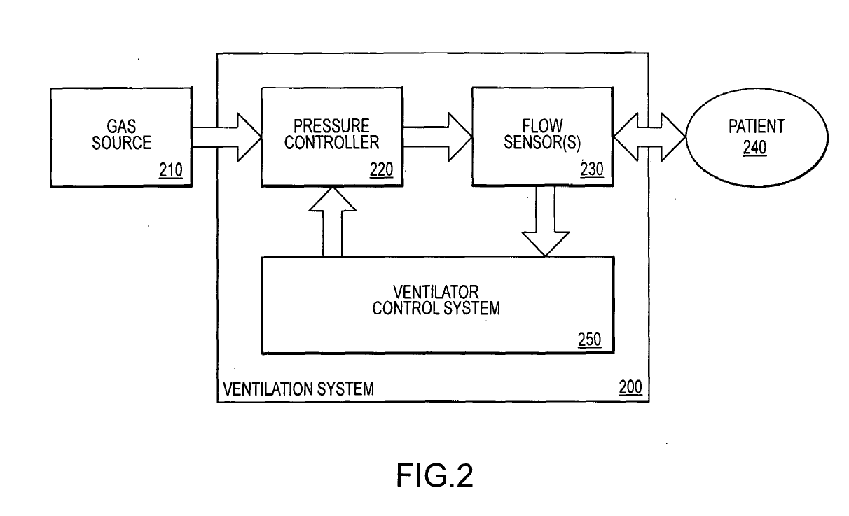

Turning to FIG. 2, a simplified block diagram of a ventilation system 200 is depicted in accordance with various embodiments of the present invention. According to this simplified illustration, ventilation system 200 includes gas flow path to deliver breathing gas from a gas source 210 to a patient 240. A pressure controller 220 and one or more flow sensors 230 are located along the gas flow path and in fluid communication with the gas source 210. Ventilation system 200 also includes a ventilator control system 250, which interacts with both the pressure controller 220 and the one or more flow sensors 230 as described in further detail below. In one embodiment, the ventilation system 200 comprises a critical care ventilator, such as an 840™ Ventilator System available from Nellcor Puritan Bennett LLC.

According to the present example, the pressure controller 220 receives a breathing gas from a gas source 210. The gas source 210 may include, but is not limited to, a helium source, an oxygen source, an air source, a heliox source and/or a gas source comprising a mixture of any of the foregoing. The pressure controller 220 causes the ventilation system 200 to automatically cycle between a higher pressure setting (e.g., positive pressure phase 110) and a lower pressure setting (e.g., release phase 120) associated with an alternating pressure ventilation mode at a predetermined flow by triggering a transition between the pressure settings based on time durations specified by the ventilator control system 250.

Gas delivered to the patient 240 and/or expiratory gas flow returning from the patient 240 to the ventilation system 200 may be measured by flow sensor(s) 230. Flow sensor(s) 230 may comprise any sensor known in the art that is capable of determining the flow of gas passing through or by the sensor. In some particular embodiments of the present invention, flow sensors(s) 230 may include a proximal flow sensor as is known in the art. In one embodiment, flow sensor(s) 230 includes two separate and independent flow sensors, a first sensor (not shown) configured to meter a flow of breathing gas delivered to the patient 240 from the ventilation 200 system and a second sensor (not shown) configured to meter expiratory gas flow returning from the patient 240 to the ventilation system 200.

According to one embodiment of the present invention, the one or more flow sensors 230 comprise a single flow sensor positioned at a port defining an entry to an airway of the patient 240. In such an embodiment, the single flow sensor may be configured to meter both a flow of breathing gas delivered to the patient 240 by the ventilation system 200 and a flow of gas returning from the patient 240 to the ventilation system 200. In one embodiment, a single flow sensor may be located at a connector (e.g., the patient wye) that joins the inspiratory and expiratory limbs of a two-limb patient circuit to the patient airway. Based on the disclosure provided herein, one of ordinary skill in the art will recognize a variety of different types of flow sensors that may be used in relation to different embodiments of the present invention.

As shown, ventilator control system 250 is coupled to both pressure controller 220 and flow sensor(s) 230. Ventilator control system 250 is operable to receive information from flow sensor(s) 230 regarding the flow of gas to or from patient 240. In one embodiment of the present invention, ventilator control system 250 automatically determines a T_{LOW} value based on the information received from the flow sensorts) 230 and based on a target percentage of PEFR. Responsive to a user command to initiate an alternating pressure ventilation mode, such as an APRV mode, and after receipt of values for each of the parameters associated with the alternating pressure ventilation mode, the ventilator control system 250 may cause the ventilation system 200 to automatically cycle among various pressure levels (e.g., PEEP_{HI} 140 and PEEP_{LOW} 130) by directing the pressure controller 220 to commence operation in accordance with pressure settings and durations for such pressure settings.

According to one embodiment, ventilation system 200 pressure is maintained by resistance of an exhaust orifice (not shown), which maintains flow-dependent pressure in the conduit and releases respiratory gas from the patient into the room. For example, the exhaust orifice may be an actively controlled exhalation valve that allows system pressure to be sustained at desired levels. Based on the disclosure provided herein, one of ordinary skill in the art will recognize a variety of different types of exhaust orifices that may be used in relation to different embodiments of the present invention. As described further below, a clinician may configure the ventilation system 200 to terminate a release phase of an alternating pressure ventilation mode at a target PEFR between approximately 20% of PEFR and approximately 75% of PEFR. In one embodiment, the pressure controller 220 is configured to actuate the exhalation valve so as to terminate the release phase at a time when the flow rate of the expiratory gas has decreased to about 25% to 50% of its absolute peak expiratory flow rate (PEFR).

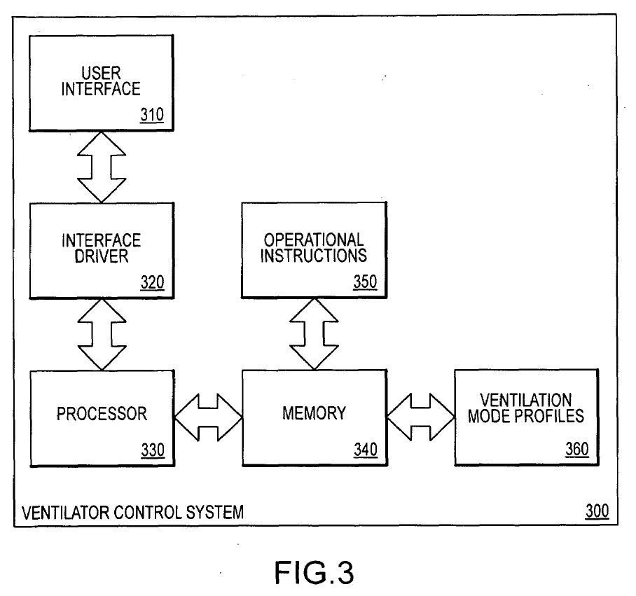

FIG. 3 depicts a ventilator control system 300 in accordance with an embodiment of the present invention that is capable of receiving information and/or parameters regarding various ventilation modes, receiving information from one or more flow sensors and governing the configuration of an alternating pressure ventilation mode based on an automatically determined duration at a lower pressure setting. Ventilator control system 300 includes a user interface 310 that is controlled by a processor 330 via an interface driver 320. In some embodiments of the present invention, user interface 310 is a touch screen interface that is capable of receiving user commands that are provided to processor 330, and is capable of providing a user display based on information provided from processor 330. It should be noted that the aforementioned touch screen user interface is merely exemplary, and that one of ordinary skill in the art will recognize a variety of user interfaces that may be utilized in relation to different embodiments of the present invention.

Processor 330 may be any processor known in the art that is capable of receiving feedback from and conveying information via user interface 310, executing various operational instruction 350 maintained in a memory 340, and processing and otherwise interacting with various other input/output (I/O) devices, such as flow sensors and a pressure controller. In one embodiment of the present invention, processor 330 may receive interrupts on a periodic basis from flow sensors (e.g., flow sensor(s) 230). Such interrupts may be received, for example, whenever a change in gas flow between the ventilation system 200 and the patient 240 is detected or whenever new gas flow readings are available (e.g., every 5 ms). Such interrupts may be received using any interrupt scheme known in the art including, but not limited to, using a polling scheme where processor 330 periodically reviews an interrupt register, or using an asynchronous interrupt port of processor 330. Alternatively or additionally, the processor 330 may proactively request sensor data from flow sensors on a periodic or as needed basis. Based on the disclosure provided herein, one of ordinary skill in the art will recognize a variety of interrupt and/or polling mechanisms that may be used in relation to different embodiments of the present invention.

According to one embodiment of the present invention, processor 330 also drives the user interface 310 and responds to commands received via the user interface 310. For example, the processor 330 may generate information and/or graphics (e.g.) waveforms) indicative of, among other things, a current ventilation mode and current and historical pressure, volume and/or flow readings. The processor 330 also responds to user commands, requests and/or inputs received via the user interface 310. In one embodiment, a clinician may interact with an airway pressure versus time tracing (waveform) and/or an inspiratory and expiratory gas flow versus time tracing (waveform) to provide input to the ventilation system regarding a desired transition point between a lower pressure setting and a higher pressure setting. For example, a clinician may designate with a stylus a point on the tracing associated with a target percent of PEFR.

In one embodiment of the present invention, processor 330 also configures an alternating pressure ventilation mode by directing a pressure controller, such as pressure controller 220, based on information indicative of values of one or more APRV mode parameters, such as an indication of the higher pressure setting (e.g., the value of PEEP_{HI} in cmH₂O), an indication of the lower pressure setting (e.g., the value of PEEP_{LOW} in cmH₂O), an indication of the duration of the higher pressure setting (e.g., the value of T_{HIGH} in seconds) and an indication of the duration of the lower pressure setting (i.e., user input indicative of the target percent of PEFR at which the ventilation system should transition from the lower pressure setting to the higher pressure setting). In one embodiment, values for a subset of these parameters may be defaulted in accordance with values retrieved from stored ventilation mode profiles. Meanwhile, these and other parameter values may be manually overridden or manually initialized, respectively, by the user.

Memory 340 includes operational instructions 350 that may be software instructions, firmware instructions or some combination thereof. Operational instructions 350 are executable by processor 350, and may be used to cause processor 330 to control a ventilator in a programmed manner. In addition, according to one embodiment, memory 340 includes a number of ventilation mode profiles 360 that may identify, among other things, necessary parameters for the particular ventilation mode and default values for such parameters. In one embodiment, the default value for a PEEP_{HI} parameter of an APRV mode is between approximately 17 to 35 cmH₂O, the default value for a PEEP_{LOW} parameter is between approximately 0 to 10 cmH₂O and the default value for a THIGH parameter is approximately between 3.5 to 6.5 seconds.

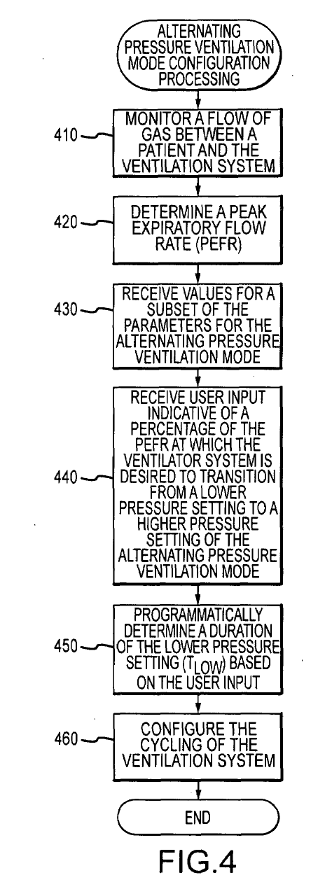

Turning now to FIG. 4., a flow diagram depicts configuration of an alternating pressure ventilation mode in accordance with an embodiment of the present invention. According to the present example, it is assumed the ventilation system has been directed to enter an APRV mode. As depicted, the process begins at block 410 in which the ventilation system commences monitoring of a flow of gas between a patent and the ventilation system. As described above, such monitoring may be performed by one or more flow sensors 230 and may meter either or both of a flow of breathing gas delivered to the patient from the ventilation system and expiratory gas flow returning from the patient to the ventilation system.

At block 420, a peak expiratory flow rate (PEFR) is determined based on the flow monitoring. According to one embodiment, the current PEFR is determined based on an average over a predetermined or specified number of sensor measurements or over a predetermined or specified number of inhalation/exhalation cycles. Alternatively, the current PEFR may take into account differences in successive measurements and the determination may be delayed until successive measurements fall within a predefined absolute value range.

At block 430, values are received for a subset of the APRV mode parameters. In accordance with one embodiment of the present invention, some but not all of the ventilation mode parameters may be initialized to predefined or configurable default values. For example, one or more of a default value for a PEEP_{HI} parameter, a default value for a PEEP_{LOW} parameter and a default value for a T_{HIGH} parameter of an APRV mode may be retrieved from a stored ventilation mode profile, such as one of ventilation mode profiles 360. Furthermore, in various embodiments of the present invention, the clinician may override the default parameter values and/or may specify or otherwise select values via the user interface for any parameters for which default values are not provided.

At block 440, user input indicative of a percentage of the PEFR at which the clinician desires the ventilation system to transition from the lower pressure setting to the higher pressure setting of the APRV mode is received. In one embodiment of the present invention, the user input comprises touch screen input designating a point on a waveform corresponding to the desired target percentage of PEFR. Alternatively, the user interface of the ventilation system may provide a range of potential or permissible target percentage of PEFR values from which the user may select. For example, a predefined set of PEFR percentages may limit selection to values between approximately 20% of PEFR and approximately 75% of PEFR. In other embodiments, the user may directly specify a numeric input corresponding to the desired target percentage of the PEFR. Based on the disclosure provided herein, one of ordinary skill in the art will recognize a variety of different input mechanisms that may be used in relation to different embodiments of the present invention.

Depending upon the clinician's goals, set up, oxygenation, ventilation, weaning, the patient's condition and/or precautions during utilization of an alternating pressure ventilation mode, various ranges or target percentages of PEFR may be selected. For example, in order to limit derecruitment in connection with a patient with restrictive lung disease (RLD), the clinician may select a target percentage of PEFR between approximately 50% and approximately 75% of PEFR. However, when a patient has acute obstructive lung disease (OLD), the clinician may select a target percentage of PEFR between approximately 25% and approximately 50% of PEFR. In other cases, the clinician may wish to configure termination of the release phase of the alternating pressure ventilation mode when the expiratory gas flow rate diminishes to between approximately 40% and approximately 55% of PEFR.

Also, it is recognized percent of PEFR is not the only way for a clinician to communicate his/her desires regarding an appropriate cycle transition. In alternative embodiments, the target may be communicated in other terms, such as a fraction or a normalized value between 0 and 10, for example, that correspond to or are otherwise indicative of a target percentage of PEFR.

At block 450, the duration of the lower pressure setting (e.g., T_{LOW} ) is automatically determined based on (i) the current PEFR value and (ii) the target percent of PEFR specified by the user or otherwise derived from input by the user. In one embodiment, T_{LOW} is calculated by measuring the time from the point at which the current PEFR occurs until the target percent of PEFR is observed based on the ongoing monitoring of block 41. In some embodiments, the T_{LOW} value may be reevaluated on a periodic basis or on demand to maintain the clinician's intent and address the issue mentioned in the background in relation to the fluctuation of the timing of the target percent of PEFR as a result of changing condition of the patient's lungs.

At block 460, the cycling of the ventilation system is configured in accordance with the ventilation mode parameters. In one embodiment, ventilator control system 250 communicates desired pressure and duration settings to pressure controller 220 to cause pressure controller 220 to automatically cycle/transition between the higher pressure setting and lower pressure setting until subsequently reconfigured.

Notably, while for purposes of illustrating a particular embodiment of the present invention, various operations for configuring an alternating pressure ventilation mode are described in a particular order, it should be appreciated that independent operations may be performed in an order other than as depicted in FIG. 4. For example, the flow monitoring of block 410 may commence at any time prior to the PEFR determination, but need not be initiated prior to receipt of parameter values in blocks 430 and 440. Furthermore, the order in which values for the ventilation mode parameters is received is of no consequence; and thus block 440 may be performed prior to block 430. Based on the disclosure provided herein, one of ordinary skill in the art will appreciate a variety of alternative orderings of the processing blocks that may be used in relation to different embodiments of the present invention.

In conclusion, the invention provides novel systems, methods and devices for configuring an alternating pressure ventilation mode of a ventilation system. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is

1. A method of controlling a ventilation system comprising:
monitoring a flow of gas between a patient and the ventilation system;
determining a peak expiratory flow rate (PEFR) based on said monitoring;
receiving information indicative of values of a plurality of parameters of an alternating pressure ventilation mode of the ventilation system, the plurality of parameters including at least a higher pressure setting, a lower pressure setting and a duration of the higher pressure setting;
receiving user input indicative of a desired percentage of the PEFR at which the ventilator system should cycle from the lower pressure setting to the higher pressure setting;
programmatically determining a duration of the lower pressure setting based on the desired percentage of the PEFR; and
configuring the ventilation system to automatically cycle between the higher pressure setting and the lower pressure setting at a predetermined flow based on the plurality of parameters and the duration of the lower pressure setting.

2. The method of claim 1, wherein the alternating pressure ventilation mode comprises an Airway Pressure Release Ventilation (APRV) mode in which a ratio of the duration of the higher pressure setting to the duration of the lower pressure setting is such that all spontaneous breathing by the patient takes place during the higher pressure setting.

3. The method of claim 1, wherein the alternating pressure ventilation mode comprises a ventilation mode in which a ratio of the duration of the higher pressure setting to the duration of the lower pressure setting is configured to allow spontaneous breathing by the patient during both the lower pressure setting and the higher pressure setting.

4. The method of claim 1, wherein said monitoring a flow of gas between a patent and the ventilation system comprises:
metering a flow of breathing gas delivered to the patient from the ventilation system via a first flow sensor; and
metering expiratory gas flow returning from the patient to the ventilation system via a second flow sensor.

5. The method of claim 1, wherein said monitoring a flow of gas between a patent and the ventilation system comprises metering, via a single sensor positioned at a port defining an entry to an airway of the patient, both a flow of breathing gas delivered to the patient by the ventilation system and a flow of gas returning from the patient to the ventilation system.

6. The method of claim 1, wherein said receiving information indicative of values of a plurality of parameters comprises receiving predefined default parameter values from a ventilation mode profile.

7. The method of claim 1, wherein said receiving information indicative of a plurality of parameters comprises:
receiving a first subset of parameter values as user input via a user interface of the ventilation system; and
receiving a second subset of parameter values from predefined default parameter values associated with a ventilation mode profile.

8. The method of claim 1, wherein said receiving user input indicative of a desired percentage of the PEFR comprises receiving a touch screen input associated with an inspiratory and expiratory gas flow versus time tracing depicted on a user interface of the ventilation system.

9. The method of claim 1, wherein said receiving user input indicative of a desired percentage of the PEFR comprises receiving a user selection from a predefined set or range of PEFR percentages displayed to the user via a user interface of the ventilation system.

10. The method of claim 9, wherein the predefined set of PEFR percentages are limited to values between approximately 20% of PEFR and approximately 75% of PEFR.

11. The method of claim 1, wherein said receiving user input indicative of a desired percentage of the PEFR comprises receiving a numerical input.

12. The method of claim 11, further comprising a user interface of the ventilation system alerting the user when the numerical input is outside a range of approximately 20 to approximately 75.

13. A ventilation system comprising:
a gas flow path to deliver breathing gas from a gas source to a patient;
a pressure controller located along the gas flow path and configured to cycle the ventilation system among a plurality of pressure settings;
one or more flow sensors located along the gas flow path, the one or more flow sensors configured to monitor a flow of gas between the patient and the ventilation system;
a user interface configured to display information to an end user of the ventilation system regarding airway pressure of the patient and the flow of gas and to receive information from the end user indicative of one or more values of parameters associated with an alternating pressure ventilation mode of the ventilation system or from which the one or more values can be derived;
a processor; and
a computer-readable medium having stored thereon instructions executable by the processor, which cause the processor to:
receive information from the one or more flow sensors regarding the flow of gas;
determine a peak expiratory flow rate (EFR) based on the information regarding the flow of gas;
receive values for a subset of the parameters associated with the alternating pressure ventilation mode) the subset of the parameters including a higher pressure setting, a lower pressure setting and a duration of the higher pressure setting;
receive user input via the user interface indicative of a desired percentage of the PEFR at which the ventilator system should cycle from the lower pressure setting to the higher pressure setting;
programmatically determine a duration of the lower pressure setting based on the desired percentage of the PEFR; and
cause the ventilation system to automatically cycle between the higher pressure setting and the lower pressure setting at a predetermined flow by conveying the higher pressure setting, the lower pressure setting, the duration of the higher pressure setting and the duration of the lower pressure setting to the pressure controller.

14. The ventilation system of claim 13, wherein the ventilation system comprises a critical care ventilator.

15. The ventilation system of claim 13, wherein the alternating pressure ventilation mode comprises an Airway Pressure Release Ventilation (APRV) mode.

16. The ventilation system of claim 13) wherein the alternating pressure ventilation mode comprises a BiLevel ventilation mode.

17. The ventilation system of claim 13, wherein said one or more flow sensors comprise:
a first sensor configured to meter a flow of breathing gas delivered to the patient from the ventilation system; and
a second sensor configured to meter expiratory gas flow returning from the patient to the ventilation system.

18. The ventilation system of claim 13, wherein said one or more flow sensors comprise a single flow sensor positioned at a port defining an entry to an airway of the patient, and wherein the single flow sensor is configured to meter both a flow of breathing gas delivered to the patient by the ventilation system and a flow of gas returning from the patient to the ventilation system.

19. A method of controlling a ventilation system comprising:
a step for monitoring a flow of gas between a patient and the ventilation system;
a step for determining a peak expiratory flow rate (PEFR) based on said monitoring;
a step for receiving information indicative of values of a plurality of parameters of an alternating pressure ventilation mode of the ventilation system, the plurality of parameters including at least a higher pressure setting, a lower pressure setting and a duration of the higher pressure setting;
a step for programmatically determining a duration of the lower pressure setting based on user input indicative of a percentage of the PEFR at which the user desires the ventilation system to transition from the lower pressure setting to the higher pressure setting; and
a step for configuring the ventilation system to automatically cycle between the higher pressure setting and the lower pressure setting at a pre-determined flow based on the plurality of parameters and the duration of the lower pressure setting.

20. The method of claim 19, wherein the alternating pressure ventilation mode is selected from a plurality of supported alternating pressure ventilation modes including one or more of an Airway Pressure Release Ventilation (APRV) mode and a BiLevel ventilation mode.
